(19) United States
(12) Patent Application Publication
(10) Pub. No.: US 2025/0032722 A1
(43) Pub. Date: Jan. 30, 2025

(54) Pelican Syringe Injector Device

(71) Applicant: Jessica O'Shay, Bainbridge Island, WA (US)

(72) Inventor: Jessica O'Shay, Bainbridge Island, WA (US)

(21) Appl. No.: 18/412,715

(22) Filed: Jan. 15, 2024

Related U.S. Application Data
(60) Provisional application No. 63/516,074, filed on Jul. 27, 2023.

(51) Int. Cl. A61M 5/315

(52) U.S. Cl. CPC A61M 5/31528; A61M 5/3158; A61M 2205/121

(57) ABSTRACT

A syringe injector device is disclosed for administering more viscous medications such as those containing hyaluronidase. The syringe injector device comprises a body component that is configured to retain a standard syringe. The body component also comprises a screw-like thread component that can be rotated to depress a plunger of the standard syringe. As the screw-like thread component is rotated, it depresses the plunger on the syringe, thus injecting the medication into the subcutaneous space. Further, by calculating the rotational distance and volume, a consistent administration rate can be achieved.

### CROSS-REFERENCE TO RELATED APPLICATION

The present application claims priority to, and the benefit of, U.S. Provisional Application No. 63/516,074, which was filed on Jul. 27, 2023, and is incorporated herein by reference in its entirety.

### FIELD OF THE INVENTION

The present invention relates generally to the field of syringe injector devices. More specifically, the present invention relates to a device that utilizes a screw-like thread component to depress the plunger on a syringe to administer medication at a consistent rate. Accordingly, the present disclosure makes specific reference thereto. Nonetheless, it is to be appreciated that aspects of the present invention are also equally applicable to other like applications, devices and methods of manufacture.

### BACKGROUND

By way of background, this invention relates to improvements in syringe injector devices. Generally, medications containing hyaluronidase are administered as an injection into fatty tissue, typically in the abdomen, over five minutes. However, because of the viscosity of the medication and variations between nurses giving the medication, the rate of delivery over the five minutes varies. Further, self-administration may not be possible for patients, as they may not be receiving the benefits of the medication, if improperly administered.

Additionally, even nurses and other healthcare professionals can apply to much force to the injector of a syringe, causing the prescribed does to be exceeded or to be injected too fast. These problems can lead to medical accidents. In addition, when the conventional drug injector is pulled outward instead of pressurizing the pressurizer, the drug flows back into the case so that the drug is not quantitatively injected or the patient's blood flows back.

Accordingly, there is a demand for an improved syringe injector device that allows users to rotate a screw-like thread to depress a plunger and administer medication at a consistent rate. More particularly, there is a demand for a syringe injector device that includes different thread patterns to accommodate various application rates and medication volumes.

Therefore, there exists a long felt need in the art for a syringe injector device that allows users to rotate a screw-like thread to depress the plunger on the syringe and administer medication at a consistent rate. There is also a long felt need in the art for a syringe injector device that offers a better and more effective way for nurses and other healthcare professionals to accurately administer medication through subcutaneous injections. Further, there is a long felt need in the art for a syringe injector device that provides users with a syringe injector designed to make it much easier for administering medications containing hyaluronidase. Moreover, there is a long felt need in the art for a device that includes different thread patterns that can be altered to accommodate various application rates and medication volumes. Further, there is a long felt need in the art for a syringe injector device that prevents medical accidents of injecting too much medication or injecting the medication too fast. Finally, there is a long felt need in the art for a syringe injector device that allows for a consistent, measured rate of subcutaneous injections via the rotations of the screw.

The subject matter disclosed and claimed herein, in one embodiment thereof, comprises a syringe injector device. The device is a syringe injector for administering more viscous medications such as those containing hyaluronidase. The syringe injector device comprises a body component that is configured to retain a standard syringe. The body component also comprises a screw-like thread component that can be rotated to depress a plunger of the standard syringe. Specifically, the body component resembles a caulking gun which retains a syringe. The screw-like thread component is rotated during use. As the screw-like thread component is rotated, it depresses the plunger on the syringe, thus injecting the medication into the subcutaneous space. Further, by calculating the rotational distance and volume, a consistent administration rate can be achieved. For example, one rotation every 20 seconds would result in 15 ml over 5 minutes. Further, the thread pattern can be altered to accommodate various rates/volumes. Accordingly, the syringe injector device allows for a consistent, measured rate of subcutaneous injections via the rotations of the screw-like thread component.

In this manner, the syringe injector device of the present invention accomplishes all of the forgoing objectives and provides users with a device that administers medication at a consistent rate. The device provides a syringe injector to administer medication through subcutaneous injections. The device includes different thread patterns.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a syringe injector device. The device is a syringe injector for administering more viscous medications such as those containing hyaluronidase. The syringe injector device comprises a body component that is configured to retain a standard syringe. The body component also comprises a screw-like thread component that can be rotated to depress a plunger of the standard syringe. As the screw-like thread component is rotated, it depresses the plunger on the syringe, thus injecting the medication into the subcutaneous space. Accordingly, the syringe injector device allows for a consistent, measured rate of subcutaneous injections via the rotations of the screw-like thread component.

In one embodiment, the syringe injector device is a screw-type injector which may be used for pumping or injecting a liquid, and is particularly suitable for pumping a thick, viscous liquid medication, such as but not limited to, hyaluronidase and medications containing hyaluronidase. The syringe injector device comprises a body component and a screw-like thread component which retain a standard syringe.

In one embodiment, the syringe used with the syringe injector device is a standard syringe as is known in the art and comprises a barrel with a plunger. Specifically, the barrel of the syringe has a distal end and a proximal end, wherein the distal end refers to the end that is distal to the patient in use and the proximal end refers to the end that is proximal to the patient in use, wherein the proximal end may be connected to a needle and the distal end comprises a handle component that a user can grip to depress the plunger. In this embodiment, a needle is detachably connected to the proximal end of the barrel, for injecting medication from the syringe.

Further, the plunger has an external radial seal which forms a seal with the inner wall of the syringe barrel during use. Additionally, the plunger has a connecting end and an operating end, the connecting end penetrates into the syringe barrel through the distal end and is adjacent to the proximal end, the operating end is kept outside the syringe barrel and includes a push component suitable for a user to operate and push the plunger to slide into the barrel of the syringe. Further, the radial seal is located at the connecting end and creates a piston assembly that is disposed at the connecting end and which acts to slide with the plunger in the barrel. Thus, the piston assembly slides in the barrel when the plunger moves axially, and suction can be performed when the plunger moves towards the distal end.

In one embodiment, the syringe injector device comprises a body component and a screw-like thread component. The screw-like thread component is detachably connected to the body component and once threaded in position, contacts the push component of the plunger. As the screw-like thread component is rotated, it depresses the plunger on the syringe and injects the medication within the syringe into the subcutaneous space.

In one embodiment, the body component of the syringe injector device is configured in a half-pipe design with an open cavity for retaining the syringe. Thus, the body component is formed in a cylindrical shape, with a receiving space formed therein for receiving a conventional syringe. Specifically, the body component resembles a caulking gun, as is known in the art. The body component can be any suitable shape and size as is known in the art, as long as it acts to retain a syringe. The body component can also be available in different sizes to accommodate different sizes of syringes.

In one embodiment, the body component comprises a distal end and a proximal end. The proximal end comprises a threaded sleeve fitting for accepting the screw-like thread component. The distal end comprises a semi-circular retaining component that releasably secures the syringe via contact with the handle component of the barrel. Thus, the handle component of the barrel is slid between the semi-circular retaining components, to retain the syringe within the body component. Further, the threaded sleeve fitting is threadedly engaged with the screw-like thread component and allows the screw-like thread component to be detachably connected to the proximal end of the body component. Rotating the screw-like thread component within the body component, contacts the push component of the plunger and drives the plunger to slide in an axial direction of the barrel.

In one embodiment, the screw-like thread component is configured in a screw-like design with a gripping head and a threaded shank that threads into the threaded sleeve fitting. The screw-like thread component can be any suitable shape and size as is known in art, as long as the thread component allows a user to comfortably grip and turn the thread component during use. Further, the thread pattern can be altered to accommodate various rates/volumes, depending on the wants and/or needs of a user. For example, one rotation every 20 sections would result in 15 ml of medication dispensed over 5 minutes. In one embodiment, different screw-like thread components with differing thread patterns can be included with the body component for use. Thus, the different thread patterns can be altered to accommodate various application rates and medication volumes.

In use, a syringe is filled with medication and the plunger pulled out, creating negative pressure in the barrel of the syringe. It should be noted that, if the liquid is thick, a large negative pressure is formed in the syringe. The filled syringe is then positioned in the body component and secured via the handle component of the barrel secured within the semi-circular retaining component. The screw-like thread component is then screwed into the threaded sleeve fitting, such that the threaded shank contacts the push component of the plunger. Rotation of the screw-like thread component depresses the plunger on the syringe, injecting the medication into the subcutaneous space. Thus, the device allows for a consistent, measured rate of subcutaneous injections via the rotations of the screw-like thread component. Further, the thread pattern of the thread component can be altered to accommodate various application rates and medication volumes. By calculating the rotational distance and volume, a consistent administration rate can be achieved, even if the injection is performed at home and/or by a user who is not a trained medical professional.

In one embodiment, the syringe injector device is a reusable component that can be utilized over and over with different syringes, as needed. In another embodiment, the syringe injector device is disposable after use. Further, the device can be manufactured from common molding processes and made of heat-sealable plastic or polymers, such as polypropylene or acrylonitrile-butadiene-styrene (ABS), or any other suitable material as is known in the art, such as but not limited to, acrylic, polycarbonate, polyethylene, polyethylene terephthalate, polyvinyl chloride, polystyrene, etc. Generally, the syringe injector device is also manufactured from a material that is water resistant or waterproof, or the body component comprises a coating that is water resistant or waterproof. Furthermore, the device can be made of antibacterial or antimicrobial material or the body component comprises a coating that is antibacterial or antimicrobial.

In one embodiment, a locking member is positioned on the threaded shank of the screw-like thread component. The locking member serves to prevent reverse rotation of the screw-like thread component. Thus, the locking member prevents the thread component from being reversely rotated, during use. In one embodiment, the screw-like thread component is rotated in one direction to advance the plunger of the syringe. By employing the locking member, reverse rotation is prevented and the plunger is not drawn up the syringe barrel and medication is prevented from flowing back up in the syringe.

In yet another embodiment, the syringe injector device comprises a plurality of indicia.

In yet another embodiment, a method of administering medication at a consistent rate via a syringe is disclosed. The method includes the steps of providing a syringe injector device comprising a body component and a screw-like thread component that retain a syringe and plunger. The method also comprises filling a syringe with medication. Further, the method comprises inserting the filled syringe into the body component. The method also comprises rotating the screw-like thread component which contacts the plunger and depresses it. Finally, the method comprises injecting the medication at a consistent, measured rate via the rotation of the screw-like thread component.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one embodiment of the syringe injector device of the present invention showing the device in use in accordance with the disclosed architecture;

FIG. 2 illustrates a perspective view of one embodiment of the syringe injector device of the present invention showing a syringe inserted within the body component in accordance with the disclosed architecture;

FIG. 3 illustrates a perspective view of one embodiment of the syringe injector device of the present invention showing the body component, the screw-like thread component, and a syringe in accordance with the disclosed architecture;

FIG. 4 illustrates a perspective view of one embodiment of the syringe injector device of the present invention showing the screw-like thread component within the body component in accordance with the disclosed architecture;

FIG. 5 illustrates a perspective view of one embodiment of the syringe injector device of the present invention showing the screw-like thread component threaded into the body component in accordance with the disclosed architecture; and

FIG. 6 illustrates a flowchart showing the method of administering medication at a consistent rate via a syringe in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for a syringe injector device that allows users to rotate a screw-like thread to depress the plunger on the syringe and administer medication at a consistent rate. There is also a long felt need in the art for a syringe injector device that offers a better and more effective way for nurses and other healthcare professionals to accurately administer medication through subcutaneous injections. Further, there is a long felt need in the art for a syringe injector device that provides users with a syringe injector designed to make it much easier for administering medications containing hyaluronidase. Moreover, there is a long felt need in the art for a device that includes different thread patterns that can be altered to accommodate various application rates and medication volumes. Further, there is a long felt need in the art for a syringe injector device that prevents medical accidents of injecting too much medication or injecting the medication too fast. Finally, there is a long felt need in the art for a syringe injector device that allows for a consistent, measured rate of subcutaneous injections via the rotations of the screw.

The present invention, in one exemplary embodiment, is a novel syringe injector device. The device is a syringe injector for administering more viscous medications such as those containing hyaluronidase. The syringe injector device comprises a body component that is configured to retain a standard syringe. The body component also comprises a screw-like thread component that can be rotated to depress a plunger of the standard syringe. As the screw-like thread component is rotated, it depresses the plunger on the syringe, thus injecting the medication into the subcutaneous space. Further, by calculating the rotational distance and volume, a consistent administration rate can be achieved. The present invention also includes a novel method of administering medication at a consistent rate via a syringe. The method includes the steps of providing a syringe injector device comprising a body component and a screw-like thread component that retain a syringe and plunger. The method also comprises filling a syringe with medication. Further, the method comprises inserting the filled syringe into the body component. The method also comprises rotating the screw-like thread component which contacts the plunger and depresses it. Finally, the method comprises injecting the medication at a consistent, measured rate via the rotation of the screw-like thread component.

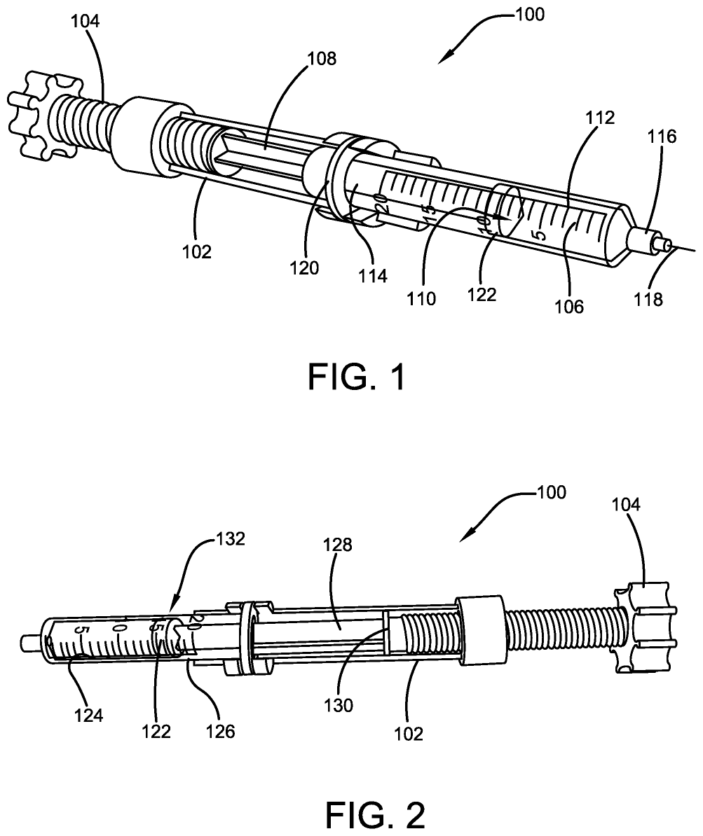

Referring initially to the drawings, FIG. 1 illustrates a perspective view of one embodiment of the syringe injector device 100 of the present invention. In the present embodiment, the syringe injector device 100 is an improved syringe injector device 100 that provides a user with a means for administering medication 110 at a consistent rate, specifically, more viscous medications such as those containing hyaluronidase. Typically, the syringe injector device 100 comprises a body component 102 with a screw-like thread component 104 that retains a syringe 106 with a plunger 108. The screw-like thread component 104 is rotated to depress a plunger 108 of the standard syringe 106, thus injecting the medication 110 into the subcutaneous space. Accordingly, the syringe injector device 100 allows for a consistent, measured rate of subcutaneous injections via the rotations of the screw-like thread component 104.

Generally, the syringe injector device 100 is a screw-type injector which may be used for pumping or injecting a liquid, and is particularly suitable for pumping a thick, viscous liquid medication 110, such as but not limited to, hyaluronidase and medications containing hyaluronidase, or any other suitable medications as is known in the art. The syringe injector device 100 comprises a body component 102 and a screw-like thread component 104 which retain a standard syringe 106.

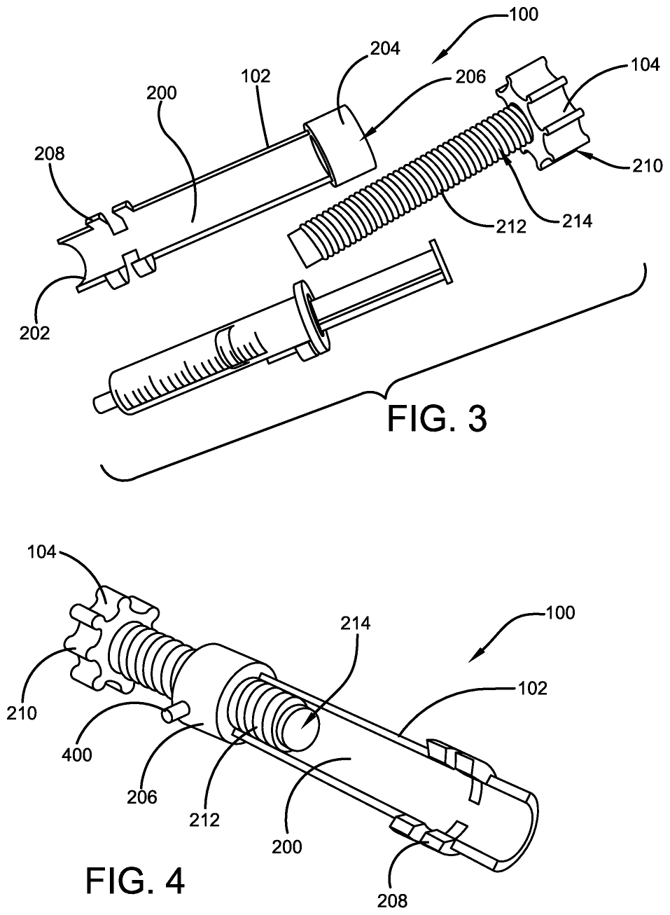

As shown in FIGS. 2-3, the syringe 106 used with the syringe injector device 100 is a standard syringe 106 as is known in the art and comprises a barrel 112 with a plunger 108. Specifically, the barrel 112 of the syringe 106 has a distal end 114 and a proximal end 116, wherein the distal end 114 refers to the end that is distal to the patient in use and the proximal end 116 refers to the end that is proximal to the patient in use, wherein the proximal end 116 may be connected to a needle 118 and the distal end 114 comprises a handle component 120 that a user can grip to depress the plunger 108. In this embodiment, a needle 118 is detachably connected to the proximal end 116 of the barrel 112, for injecting medication 110 from the syringe 106.

Furthermore, the plunger 108 has an external radial seal 122 which forms a seal with the inner wall 124 of the syringe barrel 112 during use. Additionally, the plunger 108 has a connecting end 126 and an operating end 128, the connecting end 126 penetrates into the syringe barrel 112 through the distal end 114 and is adjacent to the proximal end 116, the operating end 128 is kept outside the syringe barrel 112 and includes a push component 130 suitable for a user to operate and push the plunger 108 to slide into the barrel 112 of the syringe 106. Further, the radial seal 122 is located at the connecting end 126 and creates a piston assembly 132 that is disposed at the connecting end 126 and which acts to slide with the plunger 108 in the barrel 112. Thus, the piston assembly 132 slides in the barrel 112 when the plunger 108 moves axially, and suction can be performed when the plunger 108 moves towards the distal end 114.

Additionally, the syringe injector device 100 comprises a body component 102 and a screw-like thread component 104. The screw-like thread component 104 is detachably connected to the body component 102 and once threaded in position, contacts the push component 130 of the plunger 108. As the screw-like thread component 104 is rotated, it depresses the plunger 108 on the syringe 106 and injects the medication 110 within the syringe 106 into the subcutaneous space.

Generally, the body component 102 of the syringe injector device 100 is configured in a half-pipe design with an open cavity 200 for retaining the syringe 106. Thus, the body component 102 is formed in a cylindrical shape, with a receiving space 200 formed therein for receiving a conventional syringe 106. Specifically, the body component 102 resembles a caulking gun, as is known in the art. The body component 102 can be any suitable shape and size as is known in the art, as long as it acts to retain a syringe 106. The body component 102 can also be available in different sizes to accommodate different sizes of syringes 106, depending on the needs and/or wants of a user.

Furthermore, the body component 102 comprises a distal end 202 and a proximal end 204. The proximal end 204 comprises a threaded sleeve fitting 206 for accepting the screw-like thread component 104. The distal end 202 comprises a semi-circular retaining component 208 that releasably secures the syringe 106 via contact with the handle component 120 of the barrel 112. Thus, the handle component 120 of the barrel 112 is slid between the semi-circular retaining components 208, to retain the syringe 106 within the body component 102. Further, the threaded sleeve fitting 206 is threadedly engaged with the screw-like thread component 104 and allows the screw-like thread component 104 to be detachably connected to the proximal end 204 of the body component 102. Rotating the screw-like thread component 104 within the body component 102, contacts the push component 130 of the plunger 108 and drives the plunger 108 to slide in an axial direction of the barrel 112.

Additionally, the screw-like thread component 104 is configured in a screw-like design with a gripping head 210 and a threaded shank 212 that threads into the threaded sleeve fitting 206. The screw-like thread component 104 can be any suitable shape and size as is known in art, as long as the thread component 104 allows a user to comfortably grip and turn the thread component 104 during use. Further, the thread pattern 214 can be altered to accommodate various rates/volumes, depending on the wants and/or needs of a user. For example, one rotation every 20 sections would result in 15 ml of medication dispensed over 5 minutes. In one embodiment, different screw-like thread components 104 with differing thread patterns 214 can be included with the body component 102 for use. Thus, the different thread patterns 214 can be altered to accommodate various application rates and medication volumes.

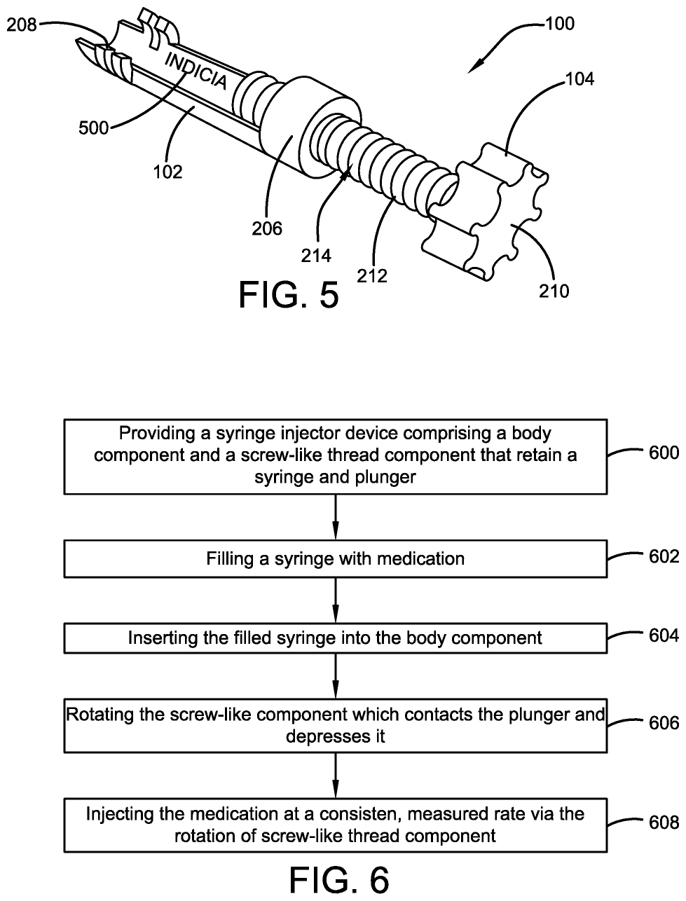

As shown in FIGS. 4-5, the syringe injector device 100 is a reusable component that can be utilized over and over with different syringes 106, as needed. In another embodiment, the syringe injector device 100 is disposable after use. Further, the device 100 can be manufactured from common molding processes and made of heat-sealable plastic or polymers, such as polypropylene or acrylonitrile-butadiene-styrene (ABS), or any other suitable material as is known in the art, such as but not limited to, acrylic, polycarbonate, polyethylene, polyethylene terephthalate, polyvinyl chloride, polystyrene, etc. Generally, the syringe injector device 100 is also manufactured from a material that is water resistant or waterproof, or the body component 102 comprises a coating that is water resistant or waterproof. Furthermore, the device 100 can be made of antibacterial or antimicrobial material or the body component 102 comprises a coating that is antibacterial or antimicrobial.

In one embodiment, a locking member 400 is positioned on the threaded shank 212 of the screw-like thread component 104. The locking member 400 serves to prevent reverse rotation of the screw-like thread component 104. Thus, the locking member 400 prevents the thread component 104 from being reversely rotated, during use. In one embodiment, the screw-like thread component 104 is rotated in one direction to advance the plunger 108 of the syringe 106. By employing the locking member 400, reverse rotation is prevented and the plunger 108 is not drawn up the syringe barrel 112 and medication 110 is prevented from flowing back up in the syringe 106.

In use, a syringe 106 is filled with medication 110 and the plunger 108 pulled out, creating negative pressure in the barrel 112 of the syringe 106. It should be noted that, if the liquid is thick, a large negative pressure is formed in the syringe 106. The filled syringe 106 is then positioned in the body component 102 and secured via the handle component 120 of the barrel 112 secured within the semi-circular retaining component 208. The screw-like thread component 104 is then screwed into the threaded sleeve fitting 206, such that the threaded shank 212 contacts the push component 130 of the plunger 108. Rotation of the screw-like thread component 104 depresses the plunger 108 on the syringe 106, injecting the medication 110 into the subcutaneous space. Thus, the device 100 allows for a consistent, measured rate of subcutaneous injections via the rotations of the screw-like thread component 104. Further, the thread pattern 214 of the thread component 104 can be altered to accommodate various application rates and medication volumes. By calculating the rotational distance and volume, a consistent administration rate can be achieved, even if the injection is performed at home and/or by a user who is not a trained medical professional.

In yet another embodiment, the syringe injector device 100 comprises a plurality of indicia 500. The body component 102 of the device 100 may include advertising, a trademark, or other letters, designs, or characters, printed, painted, stamped, or integrated into the body component 102, or any other indicia 500 as is known in the art. Specifically, any suitable indicia 500 as is known in the art can be included, such as but not limited to, patterns, logos, emblems, images, symbols, designs, letters, words, characters, animals, advertisements, brands, etc., that may or may not be syringe, medication, or brand related.

FIG. 6 illustrates a flowchart of the method of administering medication at a consistent rate via a syringe. The method includes the steps of at 600, providing a syringe injector device comprising a body component and a screw-like thread component that retain a syringe and plunger. The method also comprises at 602, filling a syringe with medication. Further, the method comprises at 604 inserting the filled syringe into the body component. The method also comprises at 606, rotating the screw-like thread component which contacts the plunger and depresses it. Finally, the method comprises at 608, injecting the medication at a consistent, measured rate via the rotation of the screw-like thread component.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “syringe injector device”, “syringe device”, “injector device”, and “device” are interchangeable and refer to the syringe injector device 100 of the present invention.

Notwithstanding the forgoing, the syringe injector device 100 of the present invention can be of any suitable size and configuration as is known in the art without affecting the overall concept of the invention, provided that it accomplishes the above stated objectives. One of ordinary skill in the art will appreciate that the syringe injector device 100 as shown in FIGS. 1-6 is for illustrative purposes only, and that many other sizes and shapes of the syringe injector device 100 are well within the scope of the present disclosure. Although the dimensions of the syringe injector device 100 are important design parameters for user convenience, the syringe injector device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A syringe injector device that provides a user with a means for administering medication at a consistent rate, the syringe injector device comprising:
a body component;
a screw-like thread component; and
a syringe;
wherein the syringe in inserted into the body component for use;
wherein the screw-like thread component is attached to an end of the body component and contacts the syringe;
wherein the screw-like thread component is rotated to depress a plunger of the syringe to inject medication from within the syringe; and
wherein the screw-like thread component allows for a consistent, measured rate of subcutaneous injections.

2. The syringe injector device of claim 1, wherein the syringe injector device is used for injecting a viscous liquid medication, such as but not limited to, hyaluronidase and medications containing hyaluronidase.

3. The syringe injector device of claim 2, wherein the syringe comprises a barrel with a plunger.

4. The syringe injector device of claim 3, wherein the barrel of the syringe has a distal end and a proximal end, wherein the proximal end is connected to a needle and the distal end comprises a handle component that a user can grip to depress the plunger.

5. The syringe injector device of claim 4, wherein the plunger has an external radial seal which forms a seal with an inner wall of the syringe barrel during use and a push component suitable for a user to operate and push the plunger to slide into the barrel.

6. The syringe injector device of claim 5, wherein the body component is configured in a half-pipe design with an open cavity for retaining the syringe.

7. The syringe injector device of claim 6, wherein the body component comprises a distal end and a proximal end, with the proximal end comprising a threaded sleeve fitting for accepting the screw-like thread component.

8. The syringe injector device of claim 7, wherein the distal end comprises a semi-circular retaining component that releasably secures the syringe via contact with the handle component of the barrel, wherein the handle component is slid between the semi-circular retaining components.

9. The syringe injector device of claim 8, wherein rotating the screw-like thread component within the body component, contacts the push component of the plunger and drives the plunger to slide in an axial direction of the barrel.

10. The syringe injector device of claim 9, wherein the screw-like thread component is configured in a screw-like design with a gripping head and a threaded shank that threads into the threaded sleeve fitting.

11. The syringe injector device of claim 10, wherein thread pattern of the screw-like thread component can be altered to accommodate various application rates and medication volumes.

12. The syringe injector device of claim 11, wherein the syringe injector device is a reusable component that can be utilized over and over with different syringes, as needed.

13. The syringe injector device of claim 12, wherein a locking member is positioned on the threaded shank of the screw-like thread component to prevent reverse rotation.

14. A syringe injector device that provides a user with a means for administering medication at a consistent rate, the syringe injector device comprising:
a body component configured in a half-pipe design with an open cavity for retaining a syringe;
a screw-like thread component configured in a screw-like design with a gripping head and a threaded shank that threads into a threaded sleeve fitting; and
a syringe comprising a barrel with a plunger;
wherein the syringe injector device is used for injecting a viscous liquid medication, such as but not limited to, hyaluronidase and medications containing hyaluronidase;
wherein the barrel of the syringe has a distal end and a proximal end, wherein the proximal end is connected to a needle and the distal end comprises a handle component that a user can grip to depress the plunger;
wherein the body component comprises a distal end and a proximal end, with the proximal end comprising the threaded sleeve fitting for accepting the screw-like thread component;
wherein the distal end comprises a semi-circular retaining component that releasably secures the syringe via contact with the handle component of the barrel, wherein the handle component is slid between the semi-circular retaining components;
wherein rotating the screw-like thread component within the body component, contacts a push component of the plunger and drives the plunger to slide in an axial direction of the barrel;
wherein the screw-like thread component is rotated to depress a plunger of the syringe to inject medication from within the syringe;
wherein the screw-like thread component allows for a consistent, measured rate of subcutaneous injections via rotations of the screw-like thread component; and
further wherein thread pattern of the screw-like thread component can be altered to accommodate various application rates and medication volumes.

15. The syringe injector device of claim 14, wherein the syringe injector device is a reusable component that can be utilized over and over with different syringes, as needed.

16. The syringe injector device of claim 14, wherein the syringe injector device is disposable after use.

17. The syringe injector device of claim 14, wherein a locking member is positioned on the threaded shank of the screw-like thread component to prevent reverse rotation.

18. The syringe injector device of claim 14 further comprising a plurality of indicia.

19. The syringe injector device of claim 14, wherein the plunger has an external radial seal which forms a seal with an inner wall of the syringe barrel during use.

20. A method of administering medication at a consistent rate via a syringe, the method comprising the following steps:
providing a syringe injector device comprising a body component and a screw-like thread component that retain a syringe and plunger;
filling a syringe with medication;
inserting the filled syringe into the body component;
rotating the screw-like thread component which contacts the plunger and depresses it; and
injecting the medication at a consistent, measured rate via the rotation of the screw-like thread component.
